(19) United States
(12) Patent Application Publication
(10) Pub. No.: US 2003/0039952 A1
(43) Pub. Date: Feb. 27, 2003

(54) Method of preparing and thawing cryopreserved cells

(75) Inventor: Tony Peled, Mevaseret Zion (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: Gamida-Cell Ltd.

(21) Appl. No.: 09/932,925

(22) Filed: Aug. 21, 2001

(51) Int. Cl. C12N 5/08

(52) U.S. Cl. 435/2; 435/372

(57) ABSTRACT

A method of preparing and thawing of cryopreserved cells without added DNase and a method of DNase-free isolation of subpopulations of thawed, cryopreserved cells which can be used to prepare and thaw Human Cord Blood cells for immunoaffinity selection and enrichment of CD34+ hematopoietic progenitor cells for expansion and transfusion.

### FIELD AND BACKGROUND OF THE INVENTION

The present invention relates to a method of preparing and thawing cryopreserved cells without the use of DNase which is traditionally used for eliminating the formation of cell aggregates, allowing for superior fluid characteristics and efficient isolation of cell subpopulations using liquid separation techniques, and more particularly, to a superior DNase-free method of preparing and thawing frozen Human Cord Blood cells for immunomagnetic selection and enrichment of CD34+ hematopoietic progenitor cells for expansion and transfusion.

Cord Blood-Derived Hematopoietic Progenitor Cells:

Hematopoietic stem cells are the cells in the bone marrow and in the peripheral blood that are able to differentiate into new blood cells. They can be used for the treatment of many congenital and acquired hematological diseases such as, for example, cancer. It is known that cord blood contains large numbers of hematopoietic stem and progenitor cells. Umbilical cord blood is an increasingly important source for stem cells, since it offers several advantages over the other stem cell sources such as bone marrow and peripheral blood in that cord blood collection is inexpensive and convenient without risk for the donor and has a low incidence of graft-versus-host disease.

Although aggressive anti-cancer treatments systematically kill tumor cells, they also destroy hematopoietic cells, and stem cells among them. Cancer patients undergoing aggressive therapy therefore require an infusion of stem cells to reconstitute their blood and immune system. Cord blood-derived hematopoietic progenitor cells have been used with increasing success as an alternative to bone marrow transplants and peripheral blood stem cell transfusions, most notably in allogenic transplantation (1).

Cryopreservation and ex-vivo Expansion of Cord Blood-Derived CD34+ Cells:

Progenitor cells are immature cells that can differentiate into red or white blood cells, or platelets; they are used to restore patients' blood and immune systems when such systems are damaged or non-functional, such as after chemotherapy or radiation as treatments for cancer. Stem cells are even earlier cells in the blood cell lineage; they have the unique capacity to self-perpetuate, termed “self-renewal”. Undifferentiated progenitor cells can be isolated using a marker on the cell known as CD34. Hematopoietic progenitor cells are rare: between 0.5-1% of bone marrow cells are progenitors carrying the CD34 marker. Stem cells are even less frequent: they comprise approximately 0.1% of all CD34+ cells (or 1 stem cell in 100,000 marrow cells). Studies on CD34+ cells have shown that CD34+ cells have a significant impact on the success of engraftment in transplantation (Benowitz, S., “Jefferson Scientists Develop Method to Isolate Blood Stem Cells, the Lifetime Source of All Blood Cells”; http://www.eurekalert.org/releases/tju-isd090199.html, Sep. 2, 1999, pp. 1-3).

Recently, standardized protocols for ex-vivo expansion of cord blood-derived hematopoietic progenitor cells (CD34+ cells) have provided a means of increasing the speed of engraftment of CD34+ cells, thereby reducing the occurrence of transplantation-related complications (7). These methods also exploit the growing availability of banked, cryopreserved cord blood as a source of typed, viable cells for expansion.

Different techniques of cord blood cryopreservation have been extensively reviewed (1-4). In 1995, Rubinstein et al. (5) established a method for standardized processing and cryopreservation of human cord blood, based on volume reduction for concentration of the leukocyte cell fraction, and rapid mixing with isotonic solutions for the minimizing of osmotic damage during cell thawing. However, with increasing experience it was found that this protocol caused severe aggregation and clumping after thawing, resulting in extensive loss of CD34+ cells during isolation of cells for expansion.

Cell aggregation and clumping is a well-documented phenomenon in thawing cryopreserved cells, mostly due to cell damage and the release of cell contents, including DNA. Donaldson et al. (6) proposed the addition of DNase to the thawed cell suspensions to increase the stem cell yield, improving the recovery of CD34+ cells from 32.5% in the absence of DNase to 76.8% with DNase in paired samples. However, with the introduction of methods for immunomagnetic separation of CD34+ cells from thawed, cryopreserved cord blood cells, the routine loss of significant numbers of the hematopoietic progenitor cells due to aggregation was noted with both the Rubinstein and DNase methods (7).

There is thus a great need for, and it would be highly advantageous to have a simple, inexpensive, DNase-free method of preparing and thawing of cryopreserved cells and a method of DNase-free isolation of subpopulations of thawed, cryopreserved cells.

### SUMMARY OF THE INVENTION

According to one aspect of the present invention there is provided a method of preparing non-aggregated cryopreserved cells for fractionation, the method comprising (a) obtaining cryopreserved cells; (b) thawing the cryopreserved cells, so as to obtain thawed cells; (c) transferring the thawed cells to a growth medium; and (d) incubating the thawed cells in the growth medium for at least several hours.

According to another aspect of the present invention there is provided a method of efficient, DNase-free isolation of subpopulations of cryopreserved blood cells, the method comprising: (a) obtaining cryopreserved cells; (b) thawing the cryopreserved cells, so as to obtain thawed cells; (c) transferring the thawed cells to a growth medium; (d) incubating the thawed cells in said growth medium for at least several hours; and (e) isolating at least one subpopulation from the thawed cells.

According to further features in preferred embodiments of the invention described below, the cryopreserved cells are thawed in a physiological buffer free of added DNase.

According to still further features in the described preferred embodiments the physiological buffer includes phosphate buffered saline, about 2.5% by volume human serum albumin, about 5% by weight dextran and about 2 mM EDTA.

According to still further features in the described preferred embodiments the growth medium includes Alpha Minimal Essential Medium.

According to still further features in the described preferred embodiments the growth medium includes Alpha Minimal Eagle's Medium and at least 2% fetal serum.

According to still further features in the described preferred embodiments the growth medium includes alpha Minimal Eagle's Medium and at least 2% autologous cord blood serum.

According to still further features in the described preferred embodiments the thawed cells are incubated in the growth medium for at least 6 hours.

According to still further features in the described preferred embodiments the thawed cells are incubated in the growth medium at a temperature between 25° C. and 43° C., preferably, about 37° C.

According to yet further features in the described preferred embodiments the cryopreserved cells are whole cord blood cells.

According to still further features in the described preferred embodiments the cryopreserved cells are a white blood cell fraction of cord blood cells.

According to yet further features in the described preferred embodiments the at least one subpopulation of thawed cells is isolated via an immunoaffinity method.

According to yet further features in the described preferred embodiments the immunoaffinity method is selected from the group consisting of immunobeads isolation, immunocolumn isolation, immunomagnetic beads isolation and fluorescent activated cell sorting.

According to still further features in the described preferred embodiments the at least one subpopulation is a CD34+ cell fraction of human and cord blood.

The present invention successfully addresses the shortcomings of the presently known configurations by providing a simple method of preparing and thawing cryopreserved cells without DNase which eliminates cell aggregation, allowing for superior fluid characteristics and efficient isolation of cell subpopulations using liquid separation techniques. More particularly, the present invention provides a superior non-aggregating, DNase-free method of preparing and thawing frozen Human Cord Blood cells for immunomagnetic selection and enrichment of CD34+ hematopoietic progenitor cells for expansion and transfusion.

### DESCRIPTION OF THE PREFERRED EMBODIMENTS

The present invention is of (i) a method of preparing and thawing cryopreserved cells without DNase; and (ii) a method of DNase-free isolation of subpopulations of thawed, cryopreserved cells which can be used to prepare and thaw Human Cord Blood cells for immunomagnetic selection and enrichment of CD34+ hematopoietic progenitor cells for expansion and transfusion.

The principles and operation of the methods according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood the phraseology and terminology employed herein is for the purpose of description and should not be regarded as limiting.

Prevention of aggregation during thawing of cryopreserved Human Cord Blood cells has both clinical and economic significance. In the last 15 years Human Cord Blood banking has become routine procedure in many countries. following the recognition of cord blood as a useful source of hematopoietic progenitor cells and as efficient vehicles for potential gene therapy in autologous and allogeneic transplant protocols. Early investigation of the conditions ensuring efficient storage of viable progenitor cells indicated that whole cord blood may be treated to remove red blood cells prior to freezing, reducing the volume required for banking of the cord blood donations. Rubinstein et al. (5) established a widely used cord blood progenitor cell banking protocol based on the isolation and cryopreservation of the cord blood leukocyte fraction. However, upon thawing cells stored by this method, significant cell damage and aggregation of the cells was consistently observed.

Cell aggregates and clumping result primarily from the release of cell contents during the cryopreservation and thawing process. Low temperature preservation of cells is generally accompanied by an ice crystallization process, which is disruptive to membranes and subcellular organelles. This process may be retarded by the use of cryopreservants. The cryopreservants in turn have negative effects on cell viability, causing cell membrane and intracellular damage due to powerful osmotic shifts, partial denaturation of protein and disorientation of cellular organelles (U.S. Pat. No. 5,776,769 to Critser, et al.). Based on measurements of CD34+ cells under osmotic stress, Woods et al. (4) recommended addition and removal of cryopreservants at ambient temperatures rather than at the lowered temperatures indicated in the Rubinstein protocol (5). The addition of DNase to the thawing medium of cord blood cells was proposed by Donaldson (6), improving the recovery rate of CD34+ progenitor cells, presumably by eliminating some cell aggregation due to the viscosity of high molecular weight DNA released from disrupted cells. However, while the addition of DNase improved the yield of viable CD34+ cells, the immunomagnetic separation of these progenitor cells from cryopreserved cells remained inefficient. A further disadvantage of the abovementioned DNase method derives from the costly requirement for recombinant human DNase (7).

While reducing the present invention to practice, it was unexpectedly realized that postconditioning thawed cryopreserved Human Cord Blood cells efficiently prevents most cell aggregation, thus eliminating the need for DNase in the thawing medium, and greatly improves the efficiency of immunomagnetic selection of CD34+ cell populations by preventing occlusion of the immunoaffinity column used for their selection When the white blood cell fraction from Human Cord Blood, cryopreserved according to standard Cord Blood Banking protocol (5) was thawed in a DNase-free medium (phosphate buffered saline (PBS)-5% Dextran-2 mM EDTA-2.5% Human Serum Albumin) and incubated overnight in growth medium containing Alpha Minimal Essential Medium and 10% Fetal Calf Serum, reduced aggregation resulting in superior flow characteristics of the resulting cell suspensions were observed. As described in the Examples section below, comparison of yields of immunomagnetic column separation of CD34+ populations from cryopreserved, thawed cells indicate that overnight postconditioning according to the method of the present invention as herein described eliminates column blockage (0% incomplete immunoseparations compared with 100% and 18.2% incomplete immunoseparations with the Rubenstein (5) and DNase (6) methods, respectively). Thus, overnight postconditioning of cells in a simple, DNase-free growth medium leads to reduced cell aggregation, greatly improving the ease and efficiency of CD34+ cell isolation for further expansion and transplantation.

The method of the present invention employs incubation of the thawed, cryopreserved cells in a growth medium, obviating the need for addition of DNase, preventing cell aggregation, thus improving the efficiency and yield of the immunomagnetic separation of CD34+ cells for ex-vivo expansion and transplantation. As described in the Examples section below, the cryopreserved leukocyte fraction of cord blood cells is thawed in PBS-dextran-EDTA-HSA and incubated overnight at a temperature of between 25° C. and 43° C., preferably about 37° C. in a growth medium, such as Alpha Minimal Eagle Medium at a concentration of preferably not more than 5×10⁶ cells per ml.

Thus, according to one aspect of the present invention there is provided a simple, inexpensive method of preparation of non-aggregated cryopreserved cells for fractionation, the method is effected by obtaining cryopreserved cells; thawing the cryopreserved cells, so as to obtain thawed cells; transferring the thawed cells to a growth medium; and incubating the thawed cells in the growth medium for at least several hours.

As used herein in the specification and in the claims section that follows, the phrase “cryopreserved cells” refers to living cells of bacterial, animal or plant origin that have been subjected to temperatures sufficiently low to cause cessation of metabolic activity. Typically, cryopreservation involves the harvesting of cells from a viable donor organism or organ, preparation of a cell suspension, introduction of a cryopreservant and rapid or gradual subjection of the cells to extreme freezing temperatures, commonly below −80° C. Examples of various techniques of freezing of different sample types are found, for example, in U.S. Pat. No. 4,004,975 to Lionetti et al., directed at freezing of human white blood cells; U.S. Pat. No. 4,890,457 to McNally et al., directed at the freezing of collagen-rich tissue, such as heart valves; U.S. Pat. No. 4,965,185 to Grischenko et al., directed to the freezing of embryos, more specifically, mammal embryos; and U.S. Pat. No. 6,140,123 to Demetriou et al., directed to cell suspensions of diverse origins.

In a preferred embodiment of the present invention, the method of cryopreservation of Human Cord Blood cells is as described by Rubinstein et al. (5), comprising volume reduction by Volex, collection of the white cell fraction and gradual freezing to −80° C. in cryopreservant consisting of 50% autologous serum, 49% saline and 1% dimethyl sulfoxide (DMSO). It will be appreciated, in the context of the present invention, that the separation of the white cell fraction and volume reduction of the aforementioned protocol are intended to minimize the volume of storage space required in Cord Blood Bank liquid nitrogen storage containers.

According to another aspect of the present invention there is provided a method of efficient, DNase-free isolation of subpopulations of cryopreserved blood cells, the method effected by obtaining cryopreserved cells; thawing the cryopreserved cells, so as to obtain thawed cells; transferring the thawed cells to a growth medium; incubating the thawed cells in the growth medium for at least several hours; and isolating at least one subpopulation from the thawed cells.

In one preferred embodiment of the invention described herein, the cryopreserved cells are thawed in a physiological buffer without added DNase. Preferably, the physiological buffer includes phosphate buffered saline (PBS), about 2.5% by volume human serum albumin, about 5% by weight dextran and about 2 mM EDTA.

As used herein in the specification and in the claims section that follows, the phrase “about” refers to a range of values from 20% more to 20% less than the designated quantity.

In still another preferred embodiment of the invention the growth medium includes Alpha Minimal Eagle's Medium. Other suitable and commercially available cell growth media include Chee's essential medium (CEM), Dulbecco's modified Eagle's medium (DMEM), Leibowitz's medium and Waymouth's medium.

Preferably, the growth medium includes Alpha Minimal Eagle's Medium and at least 2%, preferably 5%, more preferably 10% and most preferably 15% fetal serum. In this context, it will be appreciated that the fetal serum can be of any mammalian origin, such as fetal calf or fetal sheep serum, or, additionally and alternatively, artificial serum.

Most preferably the growth medium includes Alpha Minimal Eagle's Medium and at least 2%, preferably 5%, more preferably 10% and most preferably 15% autologous cord blood serum.

In one preferred embodiment of the present invention the thawed cells are incubated in the growth medium for at least 6 hours, preferably at least 9 hours, more preferably at least 12 hours and most preferably at least 18 hours.

In a preferred embodiment of the present invention described below the thawed cells are incubated in the growth medium at a temperature between 25° C. and 43° C., preferably, about 37° C.

In another preferred embodiment of the invention the cryopreserved cells are whole cord blood cells. Alternatively, and in addition, the cryopreserved cells are a white blood cell fraction of cord blood cells.

In a preferred embodiment of the invention at least one subpopulation of the thawed cells is isolated via an immunoaffinity method, such as, but not limited to, immunobeads isolation, immunocolumn isolation, immunomagnetic beads isolation and fluorescent activated cell sorting.

As used herein in the specification and the claims section that follows, the phrase “immunoaffinity method” refers to separation employing a particle or fluorescent molecule having chemically attached thereto, preferably by means of a covalent or a high affinity bond, a biologic substance capable of binding only the desired cells to the exclusion of other cells. Examples of such bonded substances include antibodies, antigens, proteins generating immune responses, nucleotides, glycoproteins, polysaccharides, lipopolysaccharides, and hormones. Descriptions of such binding effects are found in numerous publications. T cells and B cells taken from patients with systemic lupus and a variety of other rheumatologic diseases where anti-DNA antibodies are present can bind DNA, as noted in Bankhurst, A. D. and Williams, R. C. Jr., “Identification of DNA-binding Lymphocytes in Patients With Systemic Lupus Erythematosus”, Journal of Clinical Investigation, 56: 1378-1385 (1975). This property is specific to those cells that recognize DNA as an antigen, and is the case with a variety of antigens where the antigen specific T cells and B cells carry receptors for those antigens.

It has also been demonstrated that certain hematopoietic progenitor cells bind specific sugars preferentially, Aizaw, S. and Tavassoli, M., “Molecular Basis of the Recognition of Intravenously Transplanted Hemopoietic Cells by Bone Marrow,” Proceedings of the National Academy of Sciences 85: 3180-83 (1988).

Additionally, it has recently been shown that a series of proteins known as “selectins”, which also bind sugars, have been discovered in certain leukocytes, Lasky L. A. “Selectins: Interpreters of Cell-Specific Carbohydrate Information During Inflammation”. Science 258: 964-969 (1992). The selectins mediate the selective adherence of these particular leukocytes to blood vessel walls.

Methods of preparing immunomagnetic beads have been described by Hardingham et al. (Cancer Research, 53, 3455-3458 (1993)). Suitable magnetic beads are commercially available from Dynal (Oslo, Norway). The beads may be coated with any antibody capable of binding to the cells of interest. For example, isolation of hematopoietic progenitor cells may be accomplished using beads coated with antibody specific for CD34 (commercially available as CD34+ Isolation Kit, MiniMACS Separation Column and CliniMACS Separation Column from Miltenyi Biotech, Inc. and also as ISOLEX 50). Isolation of macrophages may be accomplished using antibody specific for CD14. Isolation of other cell types may be accomplished using an appropriate target cell specific antibody.

Procedures for separation may include magnetic separation, using antibody-coated magnetic beads, affinity chromatography and “panning” with antibody attached to a solid matrix, or other convenient techniques.

Techniques providing accurate separation include fluorescence activated cell sorting (FACS), which can have varying degrees of sophistication, such as multiple color channels, low angle and obtuse light scattering detecting channels, impedance channels, etc. Any technique may be employed which is not unduly detrimental to the viability of the selected cells.

Post-conditioning of cryopreserved, thawed cells by incubation in culture medium has been suggested in various cryopreservation procedures. Demetriou et al. (U.S. Pat. No. 6,140,123) propose an elaborate generalized protocol for the cryopreservation of eukaryotic cells comprising a complex nutrient cryopreservation medium, aeration of cells, high concentrations of DMSO, gradual removal of the cryopreservant and post-conditioning of thawed cells in a nutrient medium for reversal of cryopreservation stress and prevention of cell damage. However, in Demetriou et al, CD34+ cells are not specified, the components of the proposed cryopreservation medium and protocol are complex and differ greatly from the CD34+ cryopreservation methods described herein, and no mention is made of preparation of cells for further selection of subpopulations. Similarly, Chandler, et al. (U.S. Pat. No. 5,895,745) teaches incubation of cryopreserved, thawed hepatocytes in a warmed (up to 43° C.) culture media for 3 hours, with a resulting increase in metabolic activity and reduction in blebbing and ghost formation, indicating decreased cell membrane injury relative to hepatocytes thawed at 4-8° C. However, prevention of aggregation of the thawed cells and fractionation of cell subpopulations were not mentioned.

Ohta, et al. (8), working with cryopreserved, thawed peripheral blood stem cells, found that overnight culture of the thawed cells in medium containing cytokines prior to immunomagnetic isolation of CD34+ cells improved the progenitor cells' growth potential and tolerance to refreezing and thawing. The researchers made no mention of cord blood progenitor cells, and include DNase in the thawing buffer.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include cell growth and isolation techniques. Such techniques are thoroughly explained in the literature. See, for example, “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Animal Cell Culture” Freshney, R. I., ed. (1986); “Immobilized Cells and Enzymes” IRL Press, (1986); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Recovery of Hematopoietic Progenitor (CD34+) Cells from Cryopreserved Cord Blood—a Comparative Study

Materials and Methods:

Cord blood collection, preparation and cryopreservation: Whole cord blood from normal deliveries was collected by Hadassah Hospital (Jerusalem, Israel) personnel and transferred to the laboratory. Cryopreservation was performed essentially according to the method described by Rubinstein (5), employing (a) volume reduction by Volex; (b) collection of the enriched white cell fraction; and (c) gradual freezing in the presence of 50% autologous serum, 49% saline and 1% DMSO. Enriched white cell fraction aliquots were placed in Styrofoam containers at −80° C. to slow the rate of freezing. The aliquots were then transferred to liquid nitrogen for long-term storage.

Recovery of cryopreserved cord blood cells: Cells preserved as described above were thawed and treated by one of the following methods before enrichment of CD34+ cells:

DNase method (prior art): Frozen cells were thawed in a buffer (20-25 ml of buffer/6-15:10⁸ cells) containing 250 units DNase and 30 μl of 0.5 M magnesium chloride and were immediately treated to isolate CD34+ cells as described below.

Rubinstein's method (prior art): Frozen cells (20-25 ml of buffer/6-15:10⁸ cells) were thawed in 25 ml of a PBS-dextran (5%)-EDTA (2 mM)-HSA (2.5%) buffer and immediately treated to isolate CD34+ cells as described below.

The method of the present invention: Frozen cells (20-25 ml of buffer/6-15:10⁸ cells) were thawed in 25 ml of a PBS-dextran (5%)-EDTA (2 mM)-HSA (2.5%) buffer and incubated overnight at 37° C. in a growth medium (Alpha Minimal Eagle's Medium supplemented with 10% FCS) at a concentration of <5×10⁶ cells per ml. Following the overnight incubation the cells were treated to isolate CD34+ cells as described below.

Immunomagnetic isolation and recovery of CD34+ cells: Cryopreserved cord blood cells thawed by one of the above mentioned methods were centrifuged through Ficoll (Pharmacia) and purified by two passes through a CD34+ immunomagnetic isolation column (CD34+ Isolation Kit and miniMACS Separation column supplied by Miltenyi Biotec). CD34+ recovery (%) was calculated as the number of CD34+ cells eluted from the columns divided by the total number of frozen cells and multiplied by 100.

Experimental Results:

Immunomagnetic isolation of the CD34+ cells following thawing of cryopreserved cord blood is dependent on the ability of the unbound cells to move freely through the columns.

Table 1 below compares the efficiency of immunomagnetic separation of CD34+ cells following thawing of frozen cells according to three methods.

TABLE 1
Comparison of total and CD34+ cell yields using three different
frozen cord blood cells thawing methods

| | | | | Uncompleted |
|---|---|---|---|---|
| | Number of | Thawing | CD 34+ | Procedure |
| Method | Experiments^{a} | Recovery (%)^{b} | recovery (%)^{c} | (%) |
| Rubinstein | 2 | 105 ± 20.6 | 0.076 ± 0.024 | 100 |
| DNase | 14 | 71.7 ± 5.62 | 0.122 ± 0.032 | 18.2 |
| Invention | 64 | 82.3 ± 3.22 | 0.116 ± 0.008 | 0 |

^{a}Results are presented as mean ± Standard Error of Mean.
^{b}Thawing recovery was calculated as the total number of cells frozen divided by the number of cells recovered multiplied by 100.
^{c}CD34+ recovery was calculated as the number of CD34+ cells recovered after the two columns (except for the Rubinstein method where after the first column there was a total occlusion of the column by cells and debris) divided by the number of frozen cells and multiplied by 100.

Superior yield of CD34+ cells from cryopreserved cord blood cells thawed using the method of the present invention: Overnight incubation of the thawed cord blood cells prior to immunomagnetic separation according to the present invention resulted in a significant enrichment of the CD34+ cell fraction (Table 1) without column occlusion. By comparison, in both of the trials employing the Rubinstein method (Table 1), and in all other attempts using this method, there was complete occlusion of the CD34+ immunomagnetic column (100% incompleted procedures). The DNase method, although more efficient than the Rubinstein method, also resulted in column blockage causing nearly 20% incompleted procedures (Table 1). No blockage was observed using the method of the present invention (0% incompleted procedures). Considering the 100% completion rate, the CD34+ cell recovery for the thawing procedure of the present invention (0.116±0.008) was significantly better than that of the Rubinstein and DNase prior art methods (0.076±0.024 with 0% completion, and 0.122±0.032 with 81.8% completion, respectively, Table 1).

Thus, thawing of cryopreserved cord blood cells by overnight incubation in a PBS-dextran-EDTA-HSA buffer prevents occlusion of the immunomagnetic CD34+ isolation column observed with previous methods, providing a superior yield and efficient recovery of hematopoietic progenitor cells for expansion and transplantation.

It is appreciated that certain features of the invention, which are. for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES CITED

### Additional References are Cited in the Text

1. Fasoulitis S J and Schenker J G. Human umbilical cord blood banking and transplantation: a state of the art. Eur. J. Obstet. Gynecol. Reprod. Biol. May 2000; 90(1):13-25.

2. Quillen K, Berkman E M. Methods of isolation and cryopreservation of stem cells from cord blood. J. Hematother. April 1996, 5 (2):153-5.

3. Hirsch I, Claisse J P, Gluckman E. Collection, freezing, and storage of umbilical and placental cord blood. J. Hematother. 1993 Summer, 2(2):229-30.

4. Woods E J, Liu J, Derrow C W, Smith F O, Williams D A, Critser J K. Osmometric and permeability characteristics of human placental/umbilical cord blood CD34+ cells and their application to cryopreservation. J. Hematother. Stem Cell Res. April 2000, 9(2):161-73.

5. Rubinstein P, Dobrila L, Rosenfield R E, Adamson J W, Migliaccio O, Migliaccio A R, Taylor P E, Stevens C E. Processing and cryopreservation of placental/umbilical cord blood for unrelated bone marrow reconstitution. Proc. Acad. Sci. USA Oct. 24, 1995, 92(22):101 19-22.

6. Donaldson C, Armitage W J, Denning-Kendall P A, Nicol A J, Bradle B A, Hows J M. Optimal cryopreservation of human umbilical cord blood. Bone Marrow Transplant. October 1996, 18(4):725-31.

7. Querol S, Capmany G, Azqueta C, Gabarro M, Fornas O, Martin-Henao G A, Garcia J. Direct immunomagnetic method for CD34+ cell selection from cryopreserved cord blood grafts for ex vivo expansion protocols. Transfusion June 2000, 40(6):625-31.

8. Ohta K, Yamane T, Koh K R, Ohta T, Nakame H, Takubo T, Hino M, Tatsumi N. An effective method for recovering CD34 positive progenitor cells from peripheral blood stem cell apheresis products cryopreserved with simplified method. Osaka City Medical J. 1999 12, 45:2, 139-148.

What is claimed is:

1. A method of preparation of non-aggregated cryopreserved cells for fractionation, the method comprising:
(a) obtaining cryopreserved cells;
(b) thawing said cryopreserved cells, so as to obtain thawed cells;
(c) transferring said thawed cells to a growth medium; and
(d) incubating said thawed cells in said growth medium for at least several hours.

2. The method of claim 1, wherein thawing said cryopreserved cells, so as to obtain said thawed cells is in a physiological buffer free of added DNase.

3. The method of claim 2, wherein said physiological buffer includes phosphate buffered saline, about 2.5% by volume human serum albumin, about 5% by weight dextran and about 2 mM EDTA.

4. The method of claim 1, wherein said growth medium includes Alpha Minimal Eagle's Medium.

5. The method of claim 1, wherein said growth medium includes Alpha Minimal Eagle's Medium and at least 2% fetal serum.

6. The method of claim 1, wherein said growth medium includes Alpha Minimal Eagle's Medium and at least 2% autologous cord blood serum.

7. The method of claim 1, wherein said thawed cells are incubated in said growth medium for at least 6 hours.

8. The method of claim 1, wherein the thawed cells are incubated in said growth medium at a temperature between 25° C. and 43° C.

9. The method of claim 1, wherein said cryopreserved cells are whole cord blood cells.

10. The method of claim 1, wherein said cryopreserved cells are a white blood cell fraction of cord blood cells.

11. A method of efficient, DNase-free isolation of subpopulations of cryopreserved blood cells, the method comprising:
(a) obtaining cryopreserved cells;
(b) thawing said cryopreserved cells, so as to obtain thawed cells;
(c) transferring said thawed cells to a growth medium;
(d) incubating said thawed cells in said growth medium for at least several hours; and
(e) isolating at least one cell subpopulation from said thawed cells.

12. The method of claim 11, wherein thawing said cryopreserved cells, so as to obtain said thawed cells is in a physiological buffer free of added DNase.

13. The method of claim 12, wherein said physiological buffer includes phosphate buffered saline, about 2.5% by volume human serum albumin, about 5% by weight dextran and about 2 mM EDTA.

14. The method of claim 11, wherein said growth medium includes Alpha Minimal Eagle's Medium

15. The method of claim 1 1, wherein said growth medium includes Alpha Minimal Eagle's Medium and at least 2% fetal serum.

16. The method of claim 1 1, wherein said medium includes Alpha Minimal Eagle's Medium and at least 2% autologous cord blood serum.

17. The method of claim 11, wherein said thawed cells are incubated in said growth medium for at least 6 hours.

18. The method of claim 11, wherein the thawed cells are incubated in said growth medium at a temperature between 25° C. and 43° C.

19. The method of claim 11, wherein said cryopreserved cells are whole cord blood cells.

20. The method of claim 11, wherein said cryopreserved cells are a white blood cell fraction of cord blood cells.

21. The method of claim 11, wherein isolating said at least one subpopulation from said thawed cells is via an immunoaffinity method.

22. The method of claim 11, wherein said immunoaffinity method is selected from the group consisting of immunobeads isolation, immunocolumn isolation, immunomagnetic beads isolation and fluorescence activated cell sorting.

23. The method of claim 11, wherein said at least one subpopulation is a CD34+ cell fraction of human cord blood.
